(19) United States
(12) Patent Application Publication
(10) Pub. No.: US 2019/0320980 A1
(43) Pub. Date: Oct. 24, 2019

(54) FINGER CUFF WITH A BUTTERFLY SHAPE FOR NON-INVASIVE HEMODYNAMIC MEASUREMENTS

(71) Applicant: Edwards Lifesciences Corporation, Irvine, CA (US)

(72) Inventor: Jacobus Jozef Gerardus Maria Settels, De Hoef (NL)

(21) Appl. No.: 16/364,018

(22) Filed: Mar. 25, 2019

Related U.S. Application Data
(60) Provisional application No. 62/662,125, filed on Apr. 24, 2018.

(51) Int. Cl. A61B 5/00; A61B 5/022; A61B 5/0225

(52) U.S. Cl. CPC A61B 5/6843; A61B 5/02255; A61B 5/02241

(57) ABSTRACT

Disclosed is a finger cuff that is attachable to a patient's finger to be used in measuring the patient's blood pressure by a blood pressure measurement system. The finger cuff may comprise: a first end and a second end that form a butterfly shape when attached around the patient's finger; a light emitting diode (LED)-photodiode (PD) pair; and a bladder including a pair of openings. The bladder is mountable within the finger cuff such that the pair of openings surround the LED-PD pair, respectively, wherein the patient's finger surrounded in the finger cuff abuts against the bladder such that the bladder and the LED-PD pair are used in measuring the patient's blood pressure by the blood pressure measurement system.

### BACKGROUND

### Cross-Reference to Related Applications

This application claims the benefit of U.S. Provisional Patent Application No. 62/662,125 filed Apr. 24, 2018, which is incorporated by reference herein in its entirety.

### FIELD

Embodiments of the invention relate generally to non-invasive hemodynamic measurements. More particularly, embodiments of the invention relate to a finger cuff having a sensor for blood pressure measurements.

### RELEVANT BACKGROUND

Volume clamping is a technique for non-invasively measuring blood pressure in which pressure is applied to a patient's finger in such a manner that arterial pressure may be balanced by a time varying pressure to maintain a constant arterial volume. In a properly fitted and calibrated system, the applied time varying pressure should be equal to the arterial blood pressure in the finger. The applied time varying pressure may be measured to provide a reading of the patient's arterial blood pressure.

This may be accomplished by a finger cuff that is arranged or wrapped around a finger of a patient. The finger cuff may include an infrared light source, an infrared sensor, and an inflatable bladder. The infrared light may be sent through the finger in which a finger artery is present. The infrared sensor picks up the infrared light and the amount of infrared light registered by the sensor may be inversely proportional to the artery diameter.

In the finger cuff implementation, by inflating the bladder in the finger cuff, a pressure is exerted on the finger artery. If the pressure is high enough, it will compress the artery and the amount of light registered by the sensor will increase. The amount of pressure necessary in the inflatable bladder to compress the artery is dependent on the blood pressure. By controlling the pressure of the inflatable bladder, such that, the diameter of the finger artery is kept constant at its unloaded diameter, the blood pressure may be monitored in very precise detail, as the pressure in the inflatable bladder is directly linked to the blood pressure. In a typical present day finger cuff implementation, a volume clamp system is used with the finger cuff. The volume clamp system typically includes a pressure generating system and a regulating system that includes: a pump, a valve, and a pressure sensor in a closed loop feedback system that are used to clamp the arterial volume as used in the measurement of the arterial pressure. To accurately measure blood pressure, the feedback loop provides sufficient pressure generating and releasing capabilities to match the pressure oscillations of the patient's blood pressure.

Today, finger cuff based blood pressure monitoring devices generally use the same technology (e.g., photoplethysmography or similar technologies) to measure blood pressure. Unfortunately, such finger cuff devices may not be easily attachable to a patient's finger and may not be that accurate due to the finger cuff's positioning on the patient's finger. That is, attaching the finger cuff in a suboptimal way negatively influences the measurement reliability and accuracy of the volume clamp system. Moreover, there is no intrinsic guidance or limit built in present day finger cuffs to ensure that a correctly sized finger cuff is used, thereby reducing the measurement reliability and accuracy of the volume clamp system.

### BRIEF DESCRIPTION OF THE DRAWINGS

FIG. 1 is a diagram of an example of a blood pressure measurement system according to one embodiment.

FIGS. 2A-2C are diagrams illustrating a finger cuff according to embodiments of the invention.

FIGS. 3A-3B are diagrams illustrating light pipes within a finger cuff according to embodiments of the invention.

FIG. 4 is a diagram illustrating a finger cuff having a flexible circuit according to embodiments of the invention.

FIGS. 5A-5B are diagrams illustrating another finger cuff according to embodiments of the invention.

FIG. 6 is a block diagram illustrating an example environment in which embodiments of the invention may be practiced.

### DETAILED DESCRIPTION

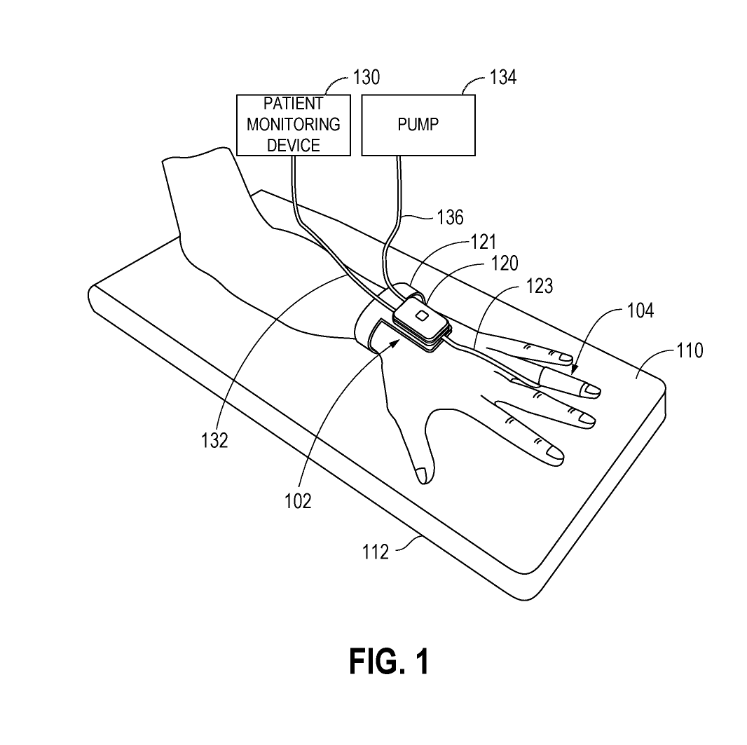

With reference to FIG. 1, which illustrates an example of a blood pressure measurement system according to one embodiment, a blood pressure measurement system 102 that includes a finger cuff 104 that may be attached to a patient's finger and a blood pressure measurement controller 120, which may be attached to the patient's body (e.g., a patient's wrist or hand) is shown.

The blood pressure measurement system 102 may further be connected to a patient monitoring device 130, and, in some embodiments, a pump 134. Further, finger cuff 104 may include a bladder (not shown) and an LED-PD pair (not shown), which are conventional for finger cuffs.

In one embodiment, the blood pressure measurement system 102 may include a pressure measurement controller 120 that includes: a small internal pump, a small internal valve, a pressure sensor, and control circuitry. In this embodiment, the control circuitry may be configured to: control the pneumatic pressure applied by the internal pump to the bladder of the finger cuff 104 to replicate the patient's blood pressure based upon measuring the volume or plethysmogram (pleth) signal received from the LED-PD pair of the finger cuff 104 (e.g., to keep the pleth signal constant). Further, the control circuitry may be configured to: control the opening of the internal valve to increase and release pneumatic pressure from the bladder; or the internal valve may simply be an orifice that is not controlled. Additionally, the control circuitry may be configured to: measure the patient's blood pressure by monitoring the pressure of the bladder based upon the input from a pressure sensor, which should be the same as patient's blood pressure, and may display the patient's blood pressure on the patient monitoring device 130.

In another embodiment, a conventional pressure generating and regulating system may be utilized, in which, a pump 134 is located remotely from the body of the patient. In this embodiment, the blood pressure measurement controller 120 receives pneumatic pressure from remote pump 134 through tube 136 and passes on the pneumatic pressure through tube 123 to the bladder of finger cuff 104. Blood pressure measurement device controller 120 may also control the pneumatic pressure (e.g., utilizing a controllable valve) applied to the finger cuff 104, as well as other functions. In this example, the pneumatic pressure applied by the pump 134 to the bladder of finger cuff 104 to replicate the patient's blood pressure based upon measuring the pleth signal received from the LED-PD pair of the finger cuff 104 (e.g., to keep the pleth signal constant) and measuring the patient's blood pressure by monitoring the pressure of the bladder may be controlled by the blood pressure measurement controller 120 and/or a remote computing device and/or the pump 134 and/or the patient monitoring device 130 to implement the volume clamping method. In some embodiments, a blood pressure measurement controller 120 is not used at all and there is simply a connection from tube 136 from a remote pump 134 including a remote pressure regulatory system to finger cuff 104, and all processing for the pressure generating and regulatory system, data processing, and display is performed by a remote computing device.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder of the finger cuff 104 in order to provide pneumatic pressure to the bladder for use in blood pressure measurement. Blood pressure measurement controller 120 may be coupled to the patient monitoring device 130 through a power/data cable 132. Also, in one embodiment, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104.

As can be seen in FIG. 1, in one example, the finger cuff 104 may be attached to a patient's finger and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some embodiments, a blood pressure measurement controller 120 may not be used at all. It should further be appreciated that the finger cuff 104 may be connected to a blood pressure measurement controller described herein, or a pressure generating and regulating system of any other kind, such as a pressure generating and regulating system that is located remotely from the body of the patient. Any kind of pressure generating and regulating system can be used, including but not limited to the blood pressure measurement controller, and may be described simply as a pressure generating and regulating system that may be used with a finger cuff 104 including an LED-PD pair and a bladder to implement the volume clamping method.

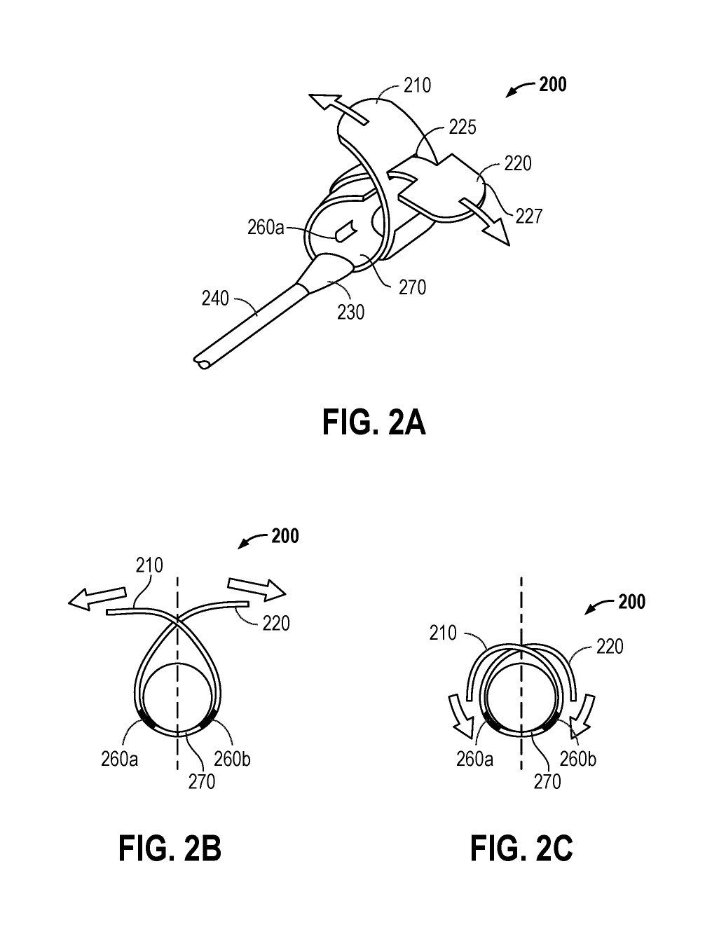

With reference to FIGS. 2A-2C, embodiments of the invention related to a finger cuff 200 will be particularly described. In some embodiments, the finger cuff 200 may be the finger cuff 104, as previously described in FIG. 1. As shown, finger cuff 200 may wrap around a patient's finger. The finger cuff 200 may be of flexible material with one or more fastening systems (e.g., a Velcro type component). As shown in FIGS. 2A-2C, finger cuff 200 may include a first end 210 and a second end 220. In one embodiment, the first end 210 may include a slot 225 and the second end 220 may include a portion 227, which may be a U-shaped portion. For attachment purposes to the patient's finger, the second end 220 (along with the portion 227) may be pulled towards the first end 210, for example by a healthcare provider, and inserted or slid through the slot 225 to form a butterfly-shaped finger cuff (e.g., butterfly flaps from first end 210 and second end 220), to wrap or attach finger cuff 200 around the patient's finger. In some embodiments, the width of portion 227 may be larger than the slot 225 to prevent the second end 220 (and portion 227) from sliding back out after it is inserted through the slot 225.

With reference to FIG. 2B, after sliding the portion 227 through the slot 225, the first end 210 and second end 220 may be pulled away from one another to a rotational position in order to apply a correct or desired tightness to the patient's finger. In applying the desired tightness to the patient's finger, the finger cuff 200 may include a built-in range limitation, for example provided by the amount of slack in the first end 210 and second end 220, that indicates whether the patient's finger is suitable (e.g., too small, too large, etc.) for the finger cuff 200. The built-in range limitation of finger cuff 200 may further provide an indication of correctness (e.g., correct or incorrect) with respect to the positioning of the finger cuff 200 on the patient's finger. As an example, the inability to apply certain tightness to the patient's finger using the finger cuff 200 may provide an intuitive feedback that the finger cuff 200 is too large or too small for the patient's finger. If the finger cuff 200 is not properly fitted, a differently sized finger cuff 200 (e.g., small, medium, larger, extra-large, etc.) may be selected and utilized instead.

With reference to FIG. 2C, when the desired tightness of the finger cuff 200 is obtained, the first end 210 and second end 220 may be fastened to the exterior of the finger cuff 200 to maintain the desired tightness on the patient's finger. For example, in one embodiment, the first end 210 on the interior may include a fastening component (e.g., a Velcro type component) that connects with another fastening component (e.g., a Velcro type component) on the exterior of the finger cuff 200. Similarly, the second end 220 on the interior may also include a fastening component (e.g., a Velcro type component) that connects with the fastening component on the exterior of the finger cuff 200. In another embodiment, the fastening components of the first end 210 and second end 220 may include removable or reusable adhesive material that may be fixedly or removably attached to the exterior surface of the finger cuff 200. It should be appreciated that these are just some examples of a fastening mechanism and that any suitable type may be utilized. In various embodiments, the butterfly flaps from first end 210 and second end 220 of finger cuff 200 may facilitate the pulling on both ends of the finger cuff 200, thereby facilitating a healthcare provider, for example, to apply a correct tightness to the patient's finger, apply a correct rotational positioning and obtain an accurate blood pressure measurement. In addition, the built-in range limitation of finger cuff 200 may automatically prevent placing a finger cuff that is inadequate (e.g., too small or too large) for a certain finger size.

As further shown in FIGS. 2A-2C, finger cuff 200 may include a bladder 270 and an LED-PD pair 260a-b mounted on the interior of the finger cuff 200. In one embodiment, the bladder 270 may include a pair of openings that surround the LED-PD pair 260a-b. The bladder 270 and LED-PD pair 260a-b may couple to tube or cable 240 through a fixed connector 230, which may be attached to finger cuff 200, to provide pneumatic pressure to the bladder 270, and to provide power to and receive data from the LED-PD pair 260a-b.

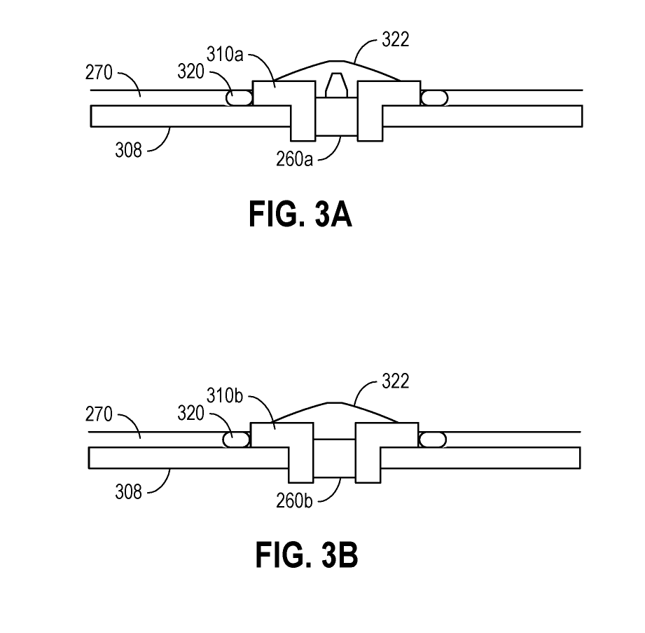

With reference to FIGS. 3A-3B, embodiments of the invention related to light pipes within a finger cuff will be particularly described. As previously described, finger cuff 200 may include bladder 270 having a pair of openings that surround the LED-PD pair 260a-b. However, in some embodiments, openings may not be present in one or both layers of the bladder 270 (e.g., the bladder 270 may be continuous) and the LED-PD pair 260a-b may simply be under the bladder 270 or under one of the layers of the bladder 270. Since the bladder 270 may be translucent, openings may not be required for optical transmission of light.

With reference to FIG. 3A, in one example, finger cuff 200 may also include a first light pipe 310a that cylindrically surrounds LED 260a and that is mounted to the backing layer 308 of the finger cuff 200, in which, the backing layer holds the bladder 270 on its inside and the LED 260a. Similarly, with reference to FIG. 3B, finger cuff 200 may also include a second light pipe 310b that cylindrically surrounds PD 260b and that is mounted to the backing layer 308 of the finger cuff 200, in which, the backing layer holds the bladder 270 on its inside and the PD 260b. As one example, the light pipes 310a and 310b may be approximately cup-shaped having a flat part and a vertical part. The flat parts extend away from the openings holding the LED 260a and PD 260b, respectively, and may abut the bladder 270, and in some examples, sealing edges 320 between the flat parts and bladder may be present, formed by the sealing process. Also, in some examples, an epoxy 322 may further seal the LED 260a and PD 260b to their respective light pipes 310a and 310b. The flat parts of the light pipes 310a and 310b may perform multiple functions, including: 1) the flat parts provide easier mounting of the LED 260a and PD 260b in the openings of the bladder 270 and backing layer 308; the flat parts in cooperation with the vertical parts operate as light pipes, as will be described in more detail hereafter; and 3) the flat parts provide a better coupling of light into the skin tissue because they constitute a flat and somewhat protruding surface for this interface, such that, the LED 260a and PD 260b protrude a bit (and therefore are not recessed, as is often the case), reducing the air gap—Any air gap between the LED and skin will generate a lot of stray light that is likely to travel around the finger (bouncing back and forth between the skin and cuff) and will not travel through it and see the artery, as is intended. The flat parts may be separate from the vertical cylindrical parts and may be referred to as guiding rings.

It should be appreciated that an objective of the light pipes 310a and 310b is to avoid stray light photons going sideways, and not going straight ahead, through the finger. Therefore, the light pipes can be me made either from absorbing material (take away stray photons), optically opaque material, or reflective material (re-routing and re-focusing stray photons). Also, an objective is to provide direct coupling, without an air gap, and without an LED or PD tilted over a certain angle, such that a positioning objective is also met. It should be appreciated that the light pipes 310a and 310b surrounding the LED and PD 206a and 260b, respectively, may serve to guide and focus light emitted from the LED 260a into a specific photon banana path extending from the LED 260a to the PD 260b and may effectively limit the photon banana width to an intended section of the finger arteries within the patient's finger in order to increase the accuracy in the blood pressure measurement. As previously described, the light from the LED 260a may travel along a specific photon banana path extending from the LED 260a to the PD 260b. In some examples, the light pipes 310a and 310b may be mounted underneath the bladder 270. In some examples, the light pipes 310a and 310b may be approximately cylindrically-shaped or of any suitable shape. In some examples, the light pipes 310a and 310b may be made from absorbing material, optically opaque material, reflective material, and/or flexible material. Although, an LED source is provided as an example of a light or optical source, it should be appreciated that any suitable LED source (red, blue, or alternative LED types) or any type of light source may utilized. As an example, a laser source utilizing a small bundle aperture could be used as a light source.

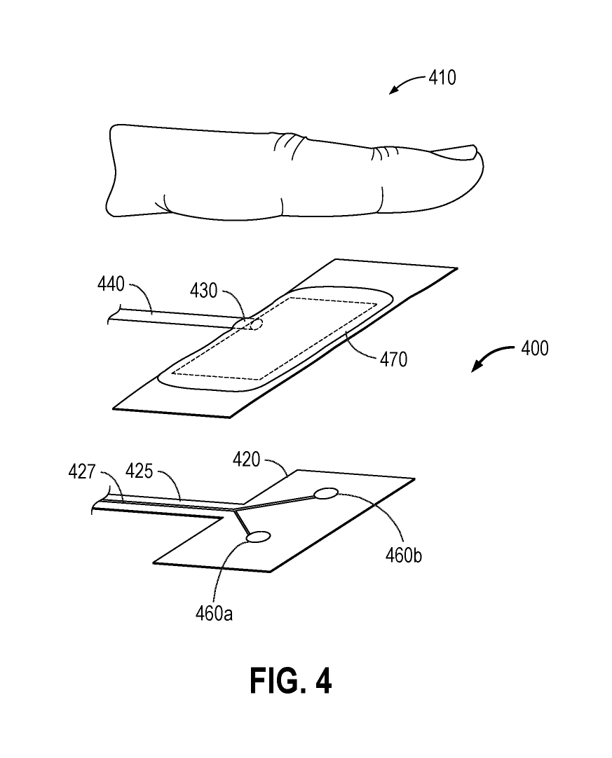

With reference to FIG. 4, embodiments of the invention related to a finger cuff 400 having a flexible circuit 420 will be particularly described. In some embodiments, the finger cuff 400 may be the finger cuff 200 of FIGS. 2A-2C. As shown, finger cuff 400 may wrap around a patient's finger 410. As illustrated in FIG. 4, finger cuff 400 may include the flexible (or flex) circuit 420 and an inflatable bladder 470 (which may be of flexible and elastic material, e.g., polyurethane). The flexible circuit 420 may be mounted on the interior of the finger cuff 400 (e.g., the wrappable portion) and the inflatable bladder 470 may be mounted over the flexible circuit 420 also onto the interior of the finger cuff (e.g., the dashed lines under the bladder 470 representing the flexible circuit 420). As illustrated in FIG. 4, flexible circuit 420 may include a pair of openings 460a-b for accommodating an LED-PD pair (e.g., LED-PD pair 260a-b), which may be electrically connectable to flexible circuit 420. Alternatively, in one embodiment the LED-PD pair may be directly mounted on the flexible circuit 420. The flexible circuit 420 may include circuitry or electronic components (not shown) that process signals (e.g., pleth signals) from the LED-PD pair and communicate the signals to another component (e.g., control circuitry as discussed in more detail herein below). In some embodiments, flexible circuit 420 may be electrically connectable to a cable 425 via signal trace (or wire) 427 to provide power to and receive data from the LED-PD pair. In addition, bladder 470 may be coupled or connectable to tube 440 via connector 430 to provide pneumatic pressure to the bladder 470.

In one embodiment, the flexible circuit 420 may be of flexible material (e.g., flexible polymer material). As can be seen in FIG. 4, the width of the flexible circuit 420 may be smaller than the full width of finger cuff 400. Further, the flexible circuit 420 may have soft and flexible edges to allow for adjustment to different finger characteristics (e.g., finger phalanx and knuckle anatomy) and may provide a tight fit and improved pressure transmission from the finger cuff 400 to the patient's finger. It should be appreciated that the flexible circuit 420 may be removably or fixedly attached to the interior of the finger cuff and similarly the bladder 470 may be removably or fixedly attached to the interior of the finger cuff. Also, in some embodiments, the flexible circuit 420 may be attached on top of the bladder 470. Accordingly, in some embodiments, flexible circuit 420 and/or bladder 470 may be physically separated or detached from one another and from the finger cuff 400.

As can be seen in FIG. 4, the flexible circuit 420 has a smaller width than the full width of the finger cuff. Further, the flexible circuit 420 may be formed to have soft, flexible edges to allow for certain adjusting to different finger phalanx and knuckle anatomy. Likewise, the inflatable bladder 470 may also have a smaller width in comparison to the full width of the finger cuff to similarly allow for certain adjusting to different finger phalanx and knuckle anatomy. Moreover, the flex circuit 420 and the inflatable bladder 470 may have reduced length in comparison to the interior of the finger cuff (e.g., the flex circuit 420 and the inflatable bladder 470 start later and end sooner than in present designs), which provide further benefits, as will be described. In particular, when the finger cuff 400 utilizes the butterfly design implementation, the more flexible material of the flex circuit 420, in combination with the butterfly design, makes it easier for a healthcare provider to obtain a good fit against the finger tissues (and therefore a correct pressure transmission from the finger cuff to the finger tissue), and ultimately to the outside of the arterial wall of the two finger arteries under the finger cuff, even on fingers with large knuckles. More particularly, flexible edges of the flex circuit 420 are applied to the patient, such that, traditional rigid edges of traditional circuits, do not sit on the patient's knuckles and cause blood vessel obstruction, nerve compression, and pain. The goodness of fit is especially crucial on the ventral side of the finger, since the two arteries are located at that side, under the bone and alongside the tendon

Further, by intentionally leaving a strip open (e.g., 5-10 mm—for a large cuff) on the dorsal side, which is achievable by the less lengthy and less wide inflatable bladder 470 and flex circuit 420, allows for (part of) the veins on the dorsal side of the finger to remain (more or less) open even during inflation of the bladder 470 to arterial pressure level. This can play a role in the prevention of blue finger tips and numbness in fingers during a prolonged measurement.

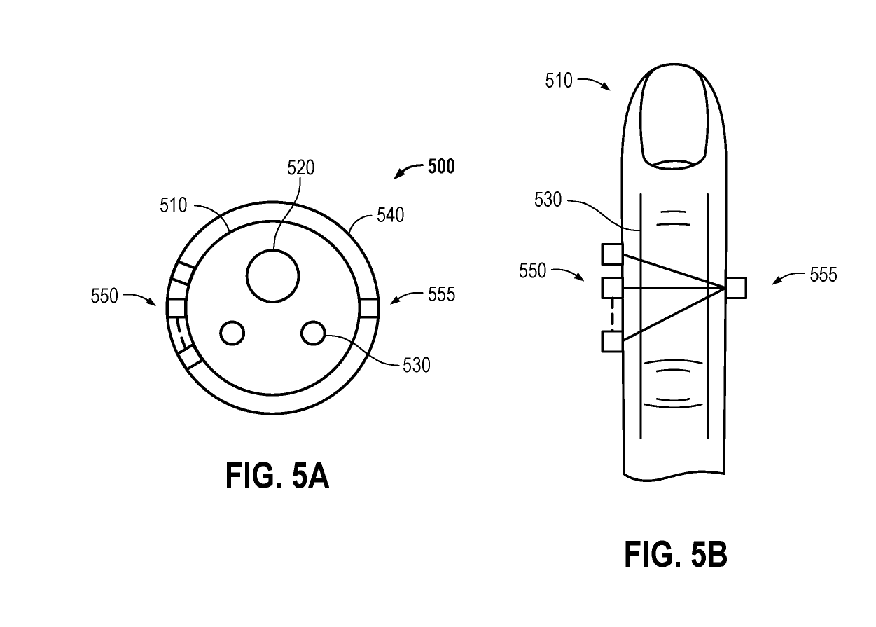

With reference to FIGS. 5A-5B, embodiments of the invention related to a finger cuff 500 will be particularly described. In some embodiments, finger cuff 500 may be the finger cuff 200 of FIG. 2A-2C or the finger cuff 400 of FIG. 4. As shown, finger cuff 500 may be wrapped around a finger 510 having finger bone 520 and finger arteries 530. In one embodiment, the finger cuff 500 may include a bladder 540, two or more LEDs 550 and a PD 555 mounted on the interior of the finger cuff 500. In one embodiment, the bladder 540 may include openings that surround the LEDs 550 and PD 555. The bladder 540, LEDs 550, and PD 555 may be coupled to a tube or cable (not shown) through a connector (also not shown), which may be attached to finger cuff 500, to provide pneumatic pressure to the bladder 540, to provide power to the LEDs 550 and to receive data from the PD 555.

Operationally, LEDs 550 may concurrently, alternatively, or in pre-defined sequences, transmit or emit light in different wavelengths and in different directions through finger arteries 530. In this scenario, the light from the LEDs 550 may be detected and registered by the PD 555 to generate a more accurate and optimal quality pleth signal, which may indicate an optimal location of the LED with respect to the location of finger arteries 530 within the patient's finger 510 for measuring the patient's blood pressure.

Further, by using more than one LED 550, additional measurements may be obtained (e.g., oxygen saturation and other physiological blood parameters, such as glucose) from signals provided by PD 555, and noise may be reduced (e.g., noise within oxygen saturation measurements).

By utilizing the previously described multiple LED 550 (two or more) volume clamp implementation, as described above, additional options are provided to measure Oxygen Saturation and other physiological blood parameter measurements during continuous blood pressure measurement, in a potentially more reliable way, as opposed to current procedures. In particular, by using the previously described multiple LED 550 volume clamp implementation, because measurements are made in a conduit artery 530, in the middle phalanx, as opposed to a capillary and arteriolar bed in the fingertip (as with current procedures), the impact of vasoconstriction (arteriolar) is reduced to a major extent. In particular, the measurement compartments can be controlled that contribute to absorption signal information, from all compartments (zero cuff pressure) to arteriolar+arterial compartment (low cuff pressure, veins collapsed) to only the arterial compartment (volume clamp at unloaded volume of arteries). In this way, much of the noise typically confounding a traditional Sp02 measurement can be taken away. In particular, during the volume clamp procedure, the blood flow in the arteries—another important confounder—is reduced to only a tiny inward and backward arterial flow, which also reduces noise compared to traditional Sp02 measurement. Further, during the volume clamp procedure, less problems exist with blood sloshing in the arteries because of motion artifacts. It should be noted that when the goal is to measure in the arterial compartment, such as the case in oxygen saturation measurements, any signal component related to tissue or arteriolar, capillary or venous compartments can be seen as noise. Also, the previously described examples combine a plurality of LEDs of different directions and wavelengths and possibly applying pressure by the bladder. Pressure in the bladder can be either constant or in a prescribed wave pattern—such as a sinus—or dynamically tracking the intra-arterial pressure as is the case during volume clamp. The purpose of the pressure thus may be two-fold: measure blood pressure and compress/collapse the compartments which may generate signal components that act as noise in the oxygen saturation measurement. Noise may be reduced as previously described utilizing a PD with multiple LEDs.

By utilizing the previously described multiple LED 550 volume clamp implementation, local oxygenation information can be measured from under the finger cuff, and this information can be used to guide an intelligent, physiology driven strategy for recommending a switch to another finger or a rest period. Therefore, this measurement system may be turned into an expert advising system based on actual information derived from the patient's local circumstances at that time.

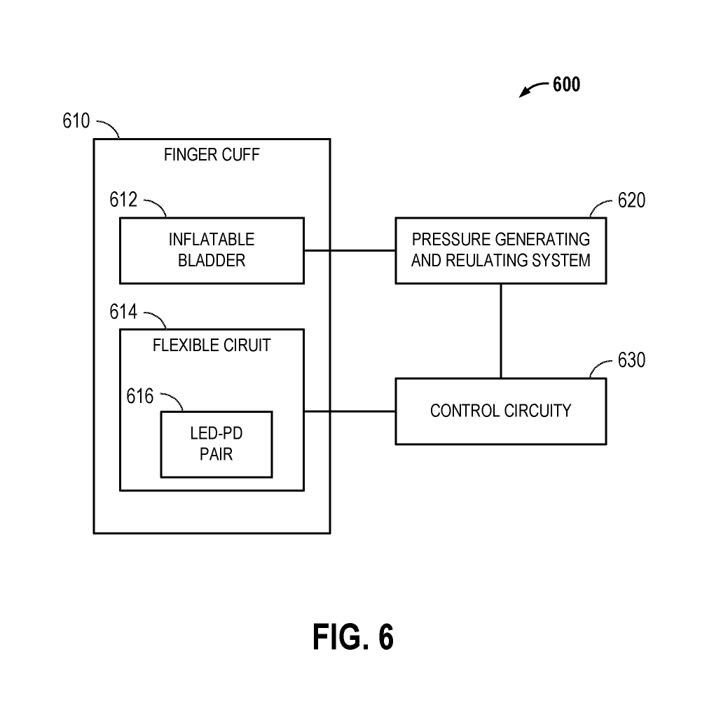

FIG. 6 is a block diagram illustrating an example environment 600 in which embodiments of the invention may be practiced. As shown, finger cuff 610 may include an inflatable bladder 612 and a flexible circuit 614. The flexible circuit 614 may be coupled or connectable to the LED-PD pair 616 to process the signals (e.g., pleth signals) from the photodiode and communicate the signals to control circuitry 630. The inflatable bladder 612 may be pneumatically connected to a pressure generating and regulating system 620. The pressure generating and regulating system 620 may generate, measure, and regulate pneumatic pressure that inflates or deflates the bladder 612, and may include elements such as a pump, a valve, a sensor, control circuitry, and/or other suitable elements. In particular, pressure generating and regulating system 620 in cooperation with control circuitry 630 may be configured to implement a volume clamp method with the finger cuff 610 by: applying pneumatic pressure to the inflatable bladder 612 of the finger cuff 610 to replicate the patient's blood pressure based upon measuring pleth signals received from the flexible circuit 614 (e.g., to keep the pleth signal constant), and measuring the patient's blood pressure by monitoring the pressure of the inflatable bladder 612 based upon input from a pressure sensor, which should be the same or correlated to the patient's blood pressure, and may further command the display of the patient's blood pressure on the patient monitoring device.

It should be appreciated that aspects of the invention previously described may be implemented in conjunction with the execution of instructions by processors, circuitry, controllers, control circuitry, etc. As an example, control circuitry may operate under the control of a program, algorithm, routine, or the execution of instructions to execute methods or processes in accordance with embodiments of the invention previously described. For example, such a program may be implemented in firmware or software (e.g. stored in memory and/or other locations) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A finger cuff attachable to a patient's finger to be used in measuring the patient's blood pressure by a blood pressure measurement system, the finger cuff comprising:
a first end and a second end that form a butterfly shape when attached around the patient's finger;
a light emitting diode (LED)-photodiode (PD) pair; and
a bladder including a pair of openings, the bladder mountable within the finger cuff such that the pair of openings surround the LED-PD pair, respectively, wherein the patient's finger surrounded in the finger cuff abuts against the bladder such that the bladder and the LED-PD pair are used in measuring the patient's blood pressure by the blood pressure measurement system.

2. The finger cuff of claim 1, wherein the first end includes a slot and the second end includes a portion that slides through the slot to form the butterfly shape when attached around the patient's finger.

3. The finger cuff of claim 2, wherein the portion slides through the slot to a rotational position to provide a built-in range limitation indicating whether the patient's finger is suitable for the finger cuff.

4. The finger cuff of claim 3, wherein the built-in range limitation automatically prevents placing an inadequate finger cuff on the patient's finger.

5. The finger cuff of claim 2, wherein the portion slides through the slot to a rotational position to provide a built-in range limitation indicating whether the finger cuff is correctly positioned on the patient's finger.

6. The finger cuff of claim 2, wherein the portion is a U-shaped portion.

7. The finger cuff of claim 6, wherein the width of the U-shaped portion is larger than the slot to prevent the U-shaped portion from sliding out after the U-shaped portion is slid through the slot.

8. The finger cuff of claim 1, wherein the first end and the second end include a fastening component that fastens to the exterior of the finger cuff in order to maintain a desired tightness on the patient's finger.

9. A method to measure a patient's blood pressure by a blood pressure measurement system utilizing a finger cuff, the finger cuff including a light emitting diode (LED)-photodiode (PD) pair and a bladder, the method comprising:
placing the finger cuff around the patient's finger such that the bladder and the LED-PD pair aid in measuring the patient's blood pressure by the blood pressure measurement system, wherein the finger cuff includes a first end and a second end;
sliding the second end through the first end to form a butterfly shape when attached around the patient's finger; and
pulling the first end and the second end away from one another to apply tightness to the patient's finger.

10. The method of claim 9, further comprising: fastening the first end and the second end to the exterior of the finger cuff to maintain the tightness on the patient's finger.

11. The method of claim 9, wherein the first end includes a slot and the second end includes a portion that slides through the slot to form the butterfly shape when attached around the patient's finger.

12. The method of claim 11, wherein sliding the second end through the first end to form the butterfly shape comprises sliding the portion through the slot to a rotational position to provide a built-in range limitation indicating whether the patient's finger is suitable for the finger cuff.

13. The method of claim 12, wherein the built-in range limitation automatically prevents placing an inadequate finger cuff on the patient's finger.

14. The method of claim 11, wherein sliding the second end through the first end to form the butterfly shape comprises sliding the portion through the slot to a rotational position to provide a built-in range limitation indicating whether the finger cuff is correctly positioned on the patient's finger.

15. The method of claim 11, wherein the portion is a U-shaped portion.

16. The method of claim 15, wherein the width of the U-shaped portion is larger than the slot to prevent the U-shaped portion from sliding out after the U-shaped portion is slid through the slot.

17. The method of claim 11, wherein the first end and the second end include a fastening component that fastens to the exterior of the finger cuff in order to maintain the tightness on the patient's finger.
